(19) United States
(12) Patent Application Publication
(10) Pub. No.: US 2024/0100298 A1
(43) Pub. Date: Mar. 28, 2024

(54) CATHETER WITH SPINE REINFORCEMENT

(71) Applicant: Neuravi Limited, Galway (IE)

(72) Inventors: Karl KEATING, Galway (IE); David VALE, Barna (IE)

(73) Assignee: Neuravi Limited, Galway (IE)

(21) Appl. No.: 17/952,595

(22) Filed: Sep. 26, 2022

(51) Int. Cl. A61M 25/00

(52) U.S. Cl. CPC A61M 25/005; A61M 25/0012; A61M 25/0045

(57) ABSTRACT

A clot retrieval catheter may have a tailored, highly flexible body section capable of navigating tortuous routes and an expandable tip for local flow restriction/arrest. The catheter may contain one or more spines extending the length of the elongate shaft which may be interwoven with the shaft braid. The distal portion of the spine may be formed into a spine hoop as part of the distal hoops of the tip section. Alternatively, the spine may form a loop around the shaft braid. Spines can be made from metal, polymer, or fiber strands. Polymer spines can be formed using a lamination or coextrusion manufacturing method. The support tube can also have a polymer jacket or membrane disposed around at least a portion of the structure.

### FIELD

The present invention generally relates devices and methods for removing acute blockages from blood vessels during intravascular medical treatments. More specifically, the present disclosure relates to a retrieval aspiration catheter with spine reinforcement.

### BACKGROUND

Aspiration and clot retrieval catheters and devices are used in mechanical thrombectomy for endovascular intervention, often in cases where patients are suffering from conditions such as acute ischemic stroke (AIS), myocardial infarction (MI), and pulmonary embolism (PE). Accessing the neurovascular bed in particular is challenging with conventional technology, as the target vessels are small in diameter, remote relative to the site of insertion, and are highly tortuous.

In delivering effective devices to the small and highly branched cerebral artery system, conventional catheters must try and balance a number of factors. The catheter must be sufficiently flexible to navigate the vasculature and endure high flexure strains, while also having the axial stiffness to offer smooth and consistent advancement along the route. Additionally, abrupt stiffness or geometric changes can hinder trackability, introduce significant stress concentrations, and increase the likelihood of device kinking or buckling.

Designs for aspirating catheters must balance the flexibility for delivery with adequate radial force and atraumatic deployment. Catheter elements must survive the severe mechanical strains imparted but also generate a sufficient radial force when expanded to prevent collapse under the suction of aspiration. Many highly flexible body designs have a reduced diameter incapable of generating the required suction force, while designs with expandable members or separate suction extensions can lack the flexibility to navigate the neurovascular. Some designs for clot retrieval catheters can have difficulty related to inadequate tensile strength. In such scenarios, the catheter can elongate or stretch during use, causing usability and durability problems. Other designs have increased the tensile strength of the catheter, but at the expensive of lateral flexibility, which make the catheter more difficult to navigate.

### SUMMARY

The present designs are aimed at providing an improved retrieval catheter with an expansile tip which incorporates features to address the above-stated deficiencies.

The designs herein can be for a spine reinforcement for a catheter capable of providing local flow restriction/arrest within the target vessel. The spine of the catheter can increase the tensile strength of the catheter while maintaining lateral flexibility, which is particularly important. This allows the catheter can be sufficiently flexible so as to be capable of navigating highly tortuous areas of the anatomy, such as the neurovascular, to reach an occlusive clot, while also having increased tensile strength. Spines can be either metal or polymer. Some spines can be interwoven with the braid. Interweaving the spines allows the spine position to be maintained during use as the product is flexed. Furthermore, this locks the spines into the braid so that the spines cannot pull through the liners or jackets.

Disclosed examples may include a catheter. The catheter may include a proximal elongate shaft which may include a longitudinal axis, a distal end, and a shaft braid. The catheter may also include a distal tip section comprising a tip braid terminating in distal hoops. Furthermore, the catheter may include a plurality of outer jackets disposed around the elongate shaft and tip section. Additionally, the catheter may include one or more metallic spines extending at least partially along the axial length of the catheter. The one or more metallic spines may inhibit tensile elongation of catheter. The catheter may include the distal hoops of the tip braid formed monolithically with wires of the shaft braid.

At least a portion of the one or more metallic spines may be a shape memory alloy.

At least a portion of the one or more metallic spines may be made of at least one of stainless steel, DFT, cobalt-chromium, titanium alloy, or tungsten.

At least one of the one or more metallic spines may be interwoven with the shaft braid.

At least one of the one or more metallic spines may have a proximal portion and a distal portion and at least a part of the proximal portion may include two or more adjacent parallel strands. The distal portion may form one or more of the distal hoops of the tip section braid.

At least a portion of the one or more metallic spines may extend the entire axial length of the catheter.

At least a portion of the one or more metallic spines may have a non-circular cross section.

The one or more metallic spines may further include a first spine and a second spine spaced 180 degrees apart.

At least a portion of the shaft braid and at least a portion of the tip braid may include different materials.

At least one of the one or more metallic spines may include sections of differing thickness.

The one or more metallic spines may be external to the shaft braid.

The one or more metallic spines may further include a single spine adhered to one of the shaft braid or the tip braid at a termination point.

Disclosed examples may include a catheter. The catheter may include an elongate tube comprising a longitudinal axis and a shaft braid which may include a first set of helical wires braided with a second set of helical wires. The catheter may also include a distal tip section comprising a tip braid with distal hoops at a distal end. Additionally, the catheter may include a plurality of outer jackets disposed around the elongate tube and tip section. Furthermore, the catheter may include one or more polymeric spines extending at least partially along the axial length of the catheter, where the polymeric spines inhibit tensile elongation of the catheter. The first set of helical wires of the shaft braid may be inverted proximally to form the second set of helical wires of the shaft braid. The distal hoops of the tip braid may be formed monolithically with the wires of the shaft braid.

At least a portion of the one or more polymeric spines may include a composition of at least one of high-density polyethylene, polyether ketone, ultra-high molecular weight polyethylene, aromatic polyamide, LCP liquid crystal polymer, Nylon or thermoset liquid-crystalline polyoxazole.

At least a portion of the one or more polymeric spines may be laminated with the outer jackets.

At least a portion of the one or more polymeric spines may be coextruded with at least one of the outer jackets.

At least one of the one or more polymeric spines may be interwoven with the shaft braid.

At least one of the one or more polymeric spines may invert through the shaft braid or the tip braid at a spine loop to form two parallel strands.

At least a portion of one of the parallel strands may extend exterior to the shaft braid and at least a portion of one of the parallel strands extending interior to the shaft braid.

Disclosed examples may include a method for constructing a catheter. The method may include arranging an inner liner around an application mandrel. The method may also include positioning one or more axial spines exterior to the outer surface of the inner liner substantially parallel to the longitudinal axis. Furthermore, the method may include disposing a braided member around at least a portion of the inner liner on the application mandrel, the braided member comprising a proximal portion and a distal portion terminating in a plurality of distal hoops. Additionally, the method may include reflowing a series of proximal outer jackets to join the catheter assembly. The method may also include loading a polymeric distal inner jacket around a reflow tool. Furthermore, the method may include removing the application mandrel and inserting the reflow tool and polymeric distal inner jacket into the distal end of catheter assembly. The method may include positioning a polymeric distal outer jacket around at least a distal portion of the catheter assembly on the reflow tool. The method may also include reflowing the polymeric distal inner jacket and polymeric distal outer jacket to the distal portion of the catheter assembly. Finally, the method may include removing the reflow tool.

The method may further include applying an inner hydrophilic coating at least the interior of the distal portion of the catheter assembly.

The method may further include utilizing a polymer in at least a portion of the one or more spines with a melt temperature in a range of approximately ±17 degrees of the melt temperature of the most distal outer jacket of the catheter. The method may also include laminating at least a portion of the one or more spines with the outer jackets. The method may also include coextruding at least a portion of the one or more spines into at least a portion of the outer jackets to form a laminar structure.

The method may include inverting at least one of the one or more spines to loop through an opening in the braided member.

The method may include utilizing a reflow tool with a flared distal end.

The method may include utilizing a polymer in at least a portion of the one or more spines with a melt temperature in a range of approximately ±6 degrees of the melt temperature of the most distal outer jacket of the catheter.

The method may include inverting a distal portion of one or more of the axial spines to form one or more of the distal hoops of the tip section braid.

An example catheter may have an elongate tube comprising a longitudinal axis and a shaft braid that comprise a first set of helical wires braided with a second set of helical wires. The catheter may also have a distal tip section comprising a tip braid with distal hoops at a distal end. Disposed around the elongate tube and tip section, the catheter may also have a plurality of outer jackets. Extending at least partially along the axial length of the catheter, there may be one or more polymeric spines. The polymeric spines may inhibit the tensile elongation of the catheter. The first set of helical wires of the shaft braid may be inverted proximally to form the second set of helical wires of the shaft braid. The distal hoops of the tip braid may be formed monolithically with the wires of the shaft braid.

An example method for constructing a catheter can include arranging an inner liner around an application mandrel. This may also include positioning one or more axial spines exterior to the outer surface of the inner liner parallel to the longitudinal axis. Furthermore, this may include disposing a braided member around at least a portion of the inner liner on the application mandrel, the braided member comprising a proximal portion and a distal portion terminating in a plurality of distal hoops. Furthermore, this may include reflowing a series of proximal outer jackets to join the catheter assembly. This may additionally include loading a polymeric distal inner jacket around a reflow tool. An additional step may include removing the application mandrel and inserting the reflow tool and polymeric distal inner jacket into the distal end of catheter assembly. Furthermore, the method may include positioning a polymeric distal outer jacket around at least a distal portion of the catheter assembly on the reflow tool. This may include reflowing the polymeric distal inner jacket and polymeric distal outer jacket to the distal portion of the catheter assembly. Additionally, this may include removing the reflow tool. Finally, this may include applying an inner hydrophilic coating at least the interior of the distal portion of the catheter assembly.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 is a side view of a catheter with a spine reinforcement with the distal portion of the spine forming one the distal hoops of the tip section braid of according to aspects of the present invention.

FIG. 2 is a side view of a catheter with spine reinforcement with one of the metallic spines interwoven with the shaft braid according to aspects of the present invention.

FIG. 3 is a sectional view of the catheter of FIG. 2 showing the position of the axial strands relative to the shaft braid according to aspects of the present invention.

FIG. 4 is a cross-sectional view of the catheter of FIG. 2 showing the position of the axial strands relative to the shaft braids according to aspects of the present invention.

FIG. 5 is a side view of a catheter with spine reinforcement containing a spine loop according to aspects of the present invention.

FIG. 6 is a cross-sectional view of the catheter of FIG. 5 showing the position of the axial strands relative to the shaft braid according to aspects of the present invention.

FIG. 7 is a side view of a catheter with spine reinforcement containing two spine loops spaced 180 degrees apart according to aspects of the present invention.

FIG. 8 is a side view of a catheter with spine reinforcement containing a single spline attached to the shaft braid according to aspects of the present invention.

FIG. 9 is an isometric view of a catheter with a spine loop with a self-expanding collapsible super bore (CSB) tip according to aspects of the present invention.

FIG. 10 is an isometric view of a catheter with a spine loop with a clot-expanding low shear tip (LST) tip according to aspects of the present invention.

FIG. 11 is a diagram showing the manufacturing of a catheter with spine reinforcement by lamination according to aspects of the present invention.

FIG. 12 is a diagram showing the manufacturing of a catheter with spine reinforcement by coextrusion according to aspects of the present invention.

FIG. 13A is front-side view of an application mandrel assembly for assembling a catheter with spine reinforcement and distal marker band according to aspects of the present invention.

FIG. 13B is a side view showing the use of the application mandrel to assemble a catheter with spine reinforcement and distal marker band according to aspects of the present invention.

FIG. 13C is an isometric view showing the use of the application mandrel to assemble a catheter with a spine loop and distal marker band according to aspects of the present invention.

FIG. 13D is an isometric view showing the use of the application mandrel to assemble a catheter with a spine hoop according to aspects of the present invention.

FIG. 13E is an isometric view of the reflow tool used to manufacture the catheter with spine reinforcement according to aspects of the present invention.

FIG. 13F is an isometric cutaway view of the reflow tool inserting the distal inner jacket into the catheter with spine reinforcement according to aspects of the present invention.

FIG. 13G is an isometric view of the reflow tool heating the distal inner jacket into the catheter with spine reinforcement according to aspects of the present invention.

FIG. 13H is an isometric view of the reflow tool being removed from the catheter with spine reinforcement according to aspects of the present invention.

FIG. 13I is a side cutaway view of a completed catheter with spine reinforcement according to aspects of the present invention.

FIGS. 14A and 14B are collectively a flow diagram showing steps to manufacture a catheter with a polymer spine reinforcement according to aspects of the present invention.

FIG. 15A is a side view showing a flat spine end of a catheter with spine reinforcement according to aspects of the present invention.

FIG. 15B is a side view showing a lasso spine end of a catheter with spine reinforcement according to aspects of the present invention.

FIG. 16 is a closeup view showing how the lasso spine end of a catheter with spine reinforcement interacts with the braid at the braid cross over point according to aspects of the present invention.

FIG. 17A is a side view of a catheter with spine reinforcement showing a spine attached by adhesive fixation according to aspects of the present invention.

FIG. 17B is a side view of a catheter with spine reinforcement showing a spine attached by outer jacket fixation according to aspects of the present invention.

FIG. 18 is a side view of a catheter with spine reinforcement where a marker band is used in attaching the spine according to aspects of the present invention.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values±20% of the recited value, e.g., “about 90%” may refer to the range of values from 71% to 99%.

As used herein, the terms “tubular” and “tube” are to be construed broadly and are not limited to a structure that is a right cylinder or strictly circumferential in cross-section or of a uniform cross-section throughout its length. For example, a tubular structure or system is generally illustrated as a substantially right cylindrical structure. However, the tubular system may have a tapered or curved outer surface without departing from the scope of the present disclosure.

An objective of at least some of the disclosed designs is to create a spine reinforcement for a catheter capable of providing both local flow restriction/arrest and high flexibility to be capable of navigating tortuous areas of the vasculature within an outer catheter to reach an occlusive clot. Such advantages can also be especially beneficial in the case of stroke intervention procedures, where vessels in the neurovascular bed are particularly small, circuitous, and fragile. As a result, a tailored axial and bending stiffness profiles of the expandable mouth tip can inhibit kinking and binding while tracking through these vessels. The tip can have a collapsed state so the clot retrieval catheter can be compatible with relatively low-profile access sheaths and outer catheters, so that a puncture wound in the patient's groin (in the case of femoral access) can be easily and reliably closed. The expandable mouth can also feature internal and/or external low-friction liners, and an outer polymer jacket or membrane disposed around the supporting structure. These improvements can lead to safe and more rapid access of a catheter and other devices to complex areas in order to more reliably remove occlusions and shorten procedure times.

Another advantage of using and having a clot retrieval catheter delivered through an outer catheter is that once the clot has entered the distal end of the clot retrieval catheter, the clot retrieval catheter can be retracted through the outer catheter such that the outer catheter is left in place to maintain access at the target treatment location. While it is appreciated that certain clots may also require that the outer catheter be retracted with the clot and inner clot retrieval catheter, the majority of clots are likely to be removed through the inner clot retrieval catheter. With this combination there will be greater confidence that the lumen of the outer catheter is clean of debris for reduced risk during contrast injection that potential thrombus remnants may be dislodged from the catheter. With traditional catheters, a user may often have to remove the outer catheter to flush any thrombus remnants outside of the body prior to injecting contrast, at the cost of losing access to the target treatment location. At least some examples of the present invention may provide means to minimize the number of catheter advancements required to treat a patient, thereby reducing the likelihood of vessel damage and the associated risk of vessel dissection in cases where multiple passes are required.

Specific examples of the present invention are now described in detail with reference to the Figures. Accessing the various vessels within the vascular, whether they are coronary, pulmonary, or cerebral, involves well-known procedural steps and use a number of conventional, commercially available accessory products. These products, such as angiographic materials, rotating hemostasis valves, and guidewires are widely used in laboratory and medical producers. When these or similar products are employed in conjunction with the system and methods of this invention in the description below, their function and exact constitution are not described in detail. The designs can often have a polymeric membrane cover/encapsulation and/or polymeric inner liner, which are typically not shown for clarity of the framework. While the description is in many cases in the context of mechanical thrombectomy treatments, the designs may be adapted for other procedures and in other body passageways as well.

Example catheters disclosed herein can include spine reinforcement. Generally, catheters with spine reinforcement can come in many varieties. The methods described herein can apply to many different types of catheters (e.g., funnel catheters or tubular aspiration catheters). The tip section of the catheter can be a variety of shapes and sizes and have a number of configurations.

The flexibility of the catheter can enable a physician to use a smaller diameter standard sheath or outer access catheter (not shown) to rapidly create a path and gain access to the vicinity of an occlusion. Example catheters disclosed herein can include an expansile tip. The expansile tip of the example catheter may be collapsed by pushing the tip into a guide catheter with the support of a tubular loading tool. The tip may then expand when it exits the guide catheter a distance from the clot. The tip may then be advanced toward the clot location. Alternatively, the expansile tip may be expanded at the treatment location to avoid having to advance an expanded tip through the vasculature, allowing the length of the elongate shaft to be relatively short.

Some example catheters disclosed herein can have an expanded deployed form of the tip section 210 at the distal end 214 of the clot retrieval catheter which can take on a flared or funnel shape. By incorporating a funnel shape in to the expansile tip, a clot can be progressively compressed during retrieval to a smaller diameter so that it can be aspirated fully through the catheter an into an aspiration syringe or canister. Because of this compression, it is less likely for firm, fibrin rich clots to become lodged in the tubular section of the clot retrieval catheter. If the clot does become lodged in the mouth of the tip, the expanded mouth will protect the clot and prevent it from dislodging as the aspiration suction is maintained and the catheter is retracted into the sheath or outer catheter.

The funnel design of the expansile tip of the disclosed examples can be an integral lattice laser cut directly and integrally with the elongate shaft of the catheter shaft. Alternately, the expansile tip lattice can be injection molded as a single piece and attached to the elongate shaft through heat welding, adhesives, or similar means. The tip section of the clot retrieval catheter can be designed to expand to a wide range of target vessel diameters, such as a carotid terminus (3.2-5.2 mm), a horizontal Ml segment of the Middle Cerebral Arteries (1.6-3.5 mm), and/or the Internal Carotid Artery (ICA, 2.7-7.5 mm). Some example catheters can be configured such that if the catheter is then retracted from an Ml segment to the ICA (or another route with a proximally increasing vessel inner diameter), the expansile tip is likely to continue to seal the vessel across a range of vessel sizes. Further, a tip capable of a range of target vessel diameters can also seal at vessel bifurcations which can have a wider cross-sectional area than the vessel proximal and vessels distal to the bifurcation. The tip section of the catheter may be expanded before reaching the treatment location or at the treatment location to avoid having to advance the expanded tip through the vasculature.

In some example catheters disclosed herein, the distal section of the aspiration clot retrieval catheter has good thrust and trackability characteristics to aid in advancing it to the target location. The catheter can therefore have multiple designs, or be fabricated from multiple materials, to give a reducing stiffness profile along the length to minimize insertion and retraction forces. In one example, the elongate shaft can be laser cut from a hypotube and formed integrally with an expanding frame portion of the tip section. In another example, the elongate shaft can be an injection molded polymer or a metal braid or weave supporting structure. Features can also be incorporated which bias bending about certain planes or encourage twisting to reduce the imparted strains. In this way the catheter will maintain excellent lateral flexibility but will not tend to expand in or kink compression.

Example catheters can also have a cover or membrane disposed around or encapsulating the elongate shaft and tip section. In the disclosed examples illustrated in the figures herein the jacket or membrane is often not shown for clarity of the underlying support structure, and the construction and appearance of such a membrane can be appreciated by those of skill in the art. Suitable membrane materials can include elastic polyurethanes such as ChronoPrene®, which can have a shore hardness of 40 A (using e.g., the ASTM D2240 A scale) or lower, or silicone elastomers. The tip section may include elastomeric polyurethanes with a percentage of silicone, such as Neusoft 42A, 52A, 62A, or 72A. Other suitable materials may include ReZalloy 40A, 50A, 60A, or 70A, Rezithane 60A or 70A, or Engage 50A. The shaft may include distal soft segments and more proximal stiffer segments, which may include non-elastomeric materials such as Pebax 25D, 35D, 45D, 55D, 63D, 72D, or 74D, or Rilasmid Nylon 12 or Rilasmid ML21. A single or variable stiffness cover can be extruded or post-formed over the elongate shaft. The cover can also be laminated, or heat welded to the structure.

Alternatively, the cover can also be a formed from a series of polymer jackets. Different jackets or sets of jackets can be disposed discrete lengths along the axis of the elongate shaft in order to give distinct pushability and flexibility characteristics to different sections of the tubular portion of the catheter. By configuring the jackets in an axial series, it is possible to transition the overall stiffness of the catheter from being stiffer at the proximal end to extremely flexible at the distal end. Alternately, the polymer jackets of the cover can be in a radial series disposed about the elongate shaft in order to tailor the material properties through the thickness. In a further example, transitions between jackets can be tapered or slotted to give a more seamless transition between flexibility profile of abutting jackets in longitudinal series.

In order to allow for smooth delivery of the clot retrieval catheter through an outer catheter, the outer surface of the membrane or outer jackets can be coated with a low-friction such as PTFE or FEP or lubricious material, such as offered by BioCoat, Surmodics, Harland, Covalon or other companies. In another example, a low-friction inner liner can also be applied to the inner circumference of the elongate shaft. Alternately, a lubricant (such as silicone oil or molybdenum disulfide) can also be used, or a coating such as a hydrophilic coating. In a further example, the inner or outer surfaces of the membrane, or the tubular section of the catheter body if formed from a polymeric extrusion, can be impregnated with a low-friction component that migrates to the surface such that the application of low-friction liners are not required.

The elongate shaft structure of the clot retrieval catheter can be of many different configurations and include spines of different configurations. The spine can be of tubular or wire construction such that it has good axial stiffness for advancing and retracting the catheter with sufficient lateral flexibility for navigating within the vascular. Use of multiple spines encourages flexing along defined planes and while reducing the possibility of the elongate shaft elongating under tensile loads, such as when the expansile tip is withdrawn into the mouth of the outer catheter.

Spines can be made from a variety of materials including metal or polymers. Metal spines can be interwoven during manufacturing of braids (which are commonly made of nitinol) and can withstand the heat setting temperature of the frame. Interweaving the spines allows the spine position to be maintained during use as the product is flexed. The spines are locked into the braid and cannot pull through liners (compared to if they were laid between liner and braid) or jackets (compared to if they were laid between jackets and braid). Liners with strike layers may be beneficial when laying the spine between the liner and the braid. PTFE liners do not melt during reflow, and therefore, the spine can block the outer jackets from bonding with the liner. If a strike layer is used (e.g., a thin 0.00025″ layer of Pebax 55D), this strike layer will also melt with the outer jackets allowing for better adhesion under the spine. Interweaving also may make assembly easier as the spines have a stable position. Metal spines can be made from nitinol, stainless steel, cobalt-chromium, titanium alloy, tungsten, or drawn filled tubing (DFT®) wire, as non-limiting examples. Tungsten spines would give the added benefit of being radiopaque. The metal spines can include a shape memory alloy. The spines may extend the entire axial length of the catheter or extend only a partial length of the catheter. Preferably, the spines extend a majority of the length of the catheter. Spines may have a non-circular cross-section and have sections of different thicknesses.

Polymer spines or fiber spines can also be used. The polymer spines can be interwoven. Metal spines may be advantageous over polymer spines when a braid requires heat setting because a polymer spine may be damaged at heat setting temperatures. Polymer spines can melt and reflow to form excellent bond with jackets and also the braid. Polymer splines can be made from high density polyethylene, poly ether ketone, ultra-high molecular weight polyethylene, aromatic polyamide, LCP liquid crystal polymer, Nylon, or thermoset liquid-crystalline polyoxazole, as non-limiting examples. The polymeric spines can be laminated or coextruded with the outer jackets. Fiber spines can be laid under or over the braid but will not melt/flow into the braid during reflow.

Referring to FIG. 1, catheter 100 is a clot retrieval catheter for use in retrieving a clot or obstruction from a vessel of a patient. Catheter 100 includes an elongate shaft 110. Elongate shaft 110 is disposed along longitudinal axis 111. The elongate shaft 110 contains an inner liner 115 and a shaft braid 120. At the proximal end (toward the right side of the drawing), the shaft braid 120 wraps around the inner liner 115. At the distal end is distal tip section 210 (toward the left side of the drawing), which can be sized and configured such that when deployed at a target site, it expands radially to atraumatically contact the inner vessel walls to provide flow restriction and arrest to prevent the unwanted aspiration of blood proximal to the tip and a large opening for aspirating and receiving the clot.

At the distal tip section 210, the shaft braid 120 expands to become the tip braid 220 and terminates in distal hoops 230. The tip braid 220 may have a portion made from different materials from the shaft braid 120. The crossing parts of the braid creates cells 226. At the distal end of the tip section 214, braid inversion 238 occurs and distal hoops 230 create the distalmost cells 228. Spine 130 extends in the direction of the longitudinal axis 111 between the layers of the shaft braid. The spine 130 is preferably metallic or polymeric. The spine 130 may be positioned to be at the cross over points of the shaft braids 120. Both strands of the spine 130 may be interwoven with the shaft braid 120 or have one strand interwoven with the shaft braid 120 and another strand laid on top of the shaft braid 120. This may be created by transition one of the braid loops into a double spine at a shaft section. Alternatively, both strands could be positioned under the shaft braid or above the shaft braid. Furthermore, one strand may be interwoven and the other strand may be positioned above or below the braid. Spine 130 contains proximal portion 143 and distal portion 144, the portions separated by transition point 135. The transition point 135 does not need to occur at the start of the inner liner 115. The transition point 135 may be closer to the proximal end of the catheter, which may create a section of shaft that is more flexible between the tip and the spine 130. The distal portion 144 of the spine creates a spine hoop 231. The spine hoop 231 may be positioned among the distal hoops 230 of the tip braid 220, so that the spine hoop 231 adds a hoop to the tip braid.

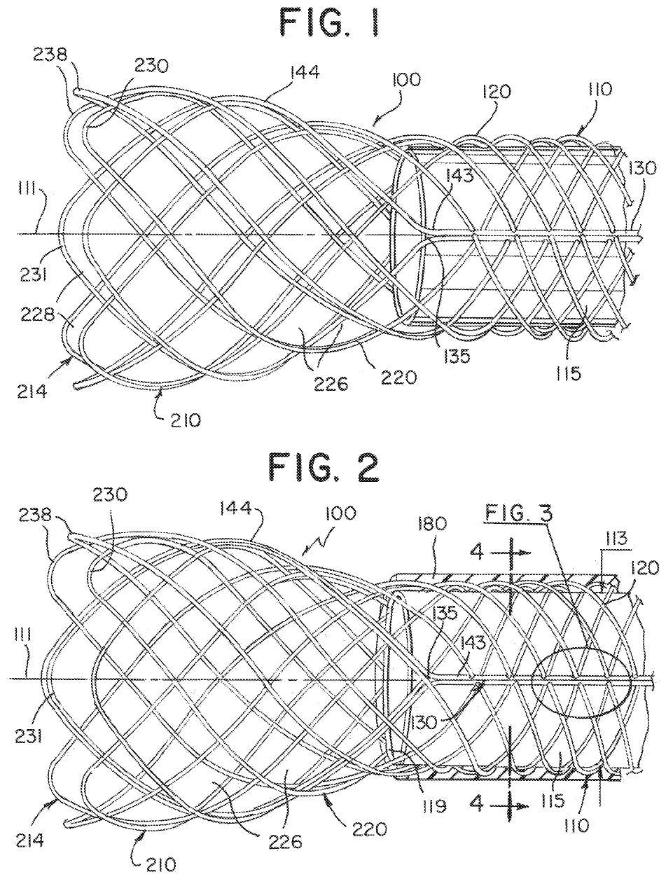

FIG. 2 is similar to FIG. 1, except that the spine hoop 231 is positioned adjacent a distal hoop 230 of the tip braid, so that the spine hoop 231 does not add an additional hoop to the tip braid, but rather reinforces one of the distal hoops 230. Referring to FIG. 2, spine 130 has proximal portion 143 and distal portion 144. The two strands of the spine are separated at transition point 135. The spine distal portion 144 may be interwoven with the shaft braid 120 such that the spine hoop 231 wraps around a distal hoop 230 of the tip braid 220. This may be created by adding an additional braid loop beside one of the existing loops.

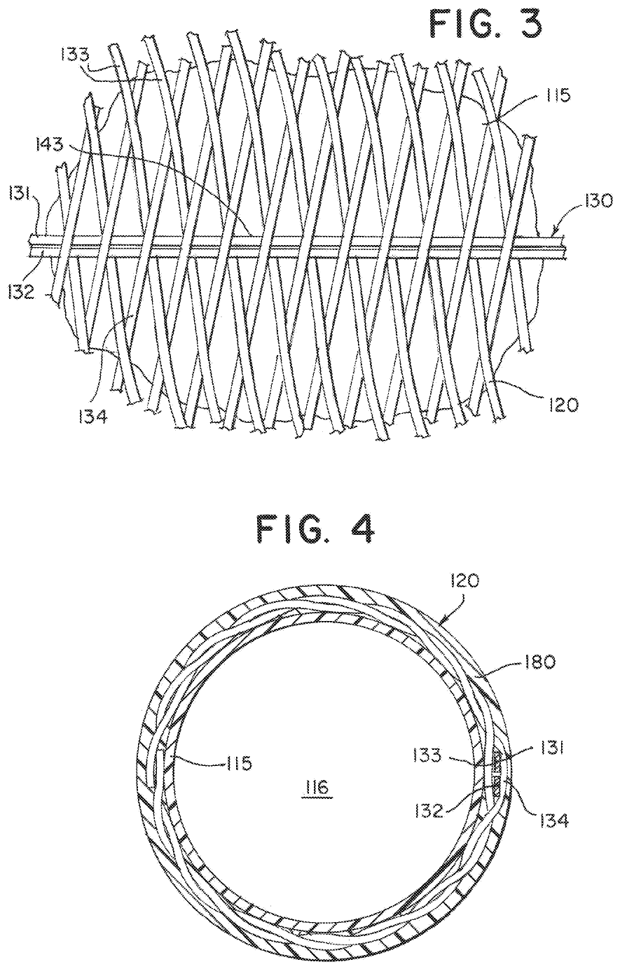

FIG. 3 shows the positioning of the spine 130 within the shaft braid 120 of the catheter of FIG. 2. The spine may have two or more adjacent parallel strands (131, 132). The proximal portion of the spine 143 extends longitudinally and contains an axial first strand 131 and an axial second strand 132. The axial first strand 131 is positioned next to the axial second strand 132 horizontally, for example at a similar radius from longitudinal axis 111 (not pictured) but may be placed in different configurations. Overlying weave strands 134 of the shaft braid 120 extend above the spine 130. Underlying weave strands 133 extend below the spine 130. The spine 130 may be positioned near a cross-over point of the weave strands 133, 134.

FIG. 4 shows a cross-sectional view of the elongate shaft 110 including spine 130 of the catheter of FIG. 2. The interior of the catheter is referred to as the inner lumen 116. The inner liner 115 surrounds the inner lumen and is below the shaft braid 120. The shaft braid 120 contains the underlying weave strands which are below the axial first strand 131 and axial second strand 132 of the spine 130. The overlying weave strands 134 are above the axial first strand 131 and axial second strand 132. The outer jacket 180 surrounds the overlying weave strands 134.

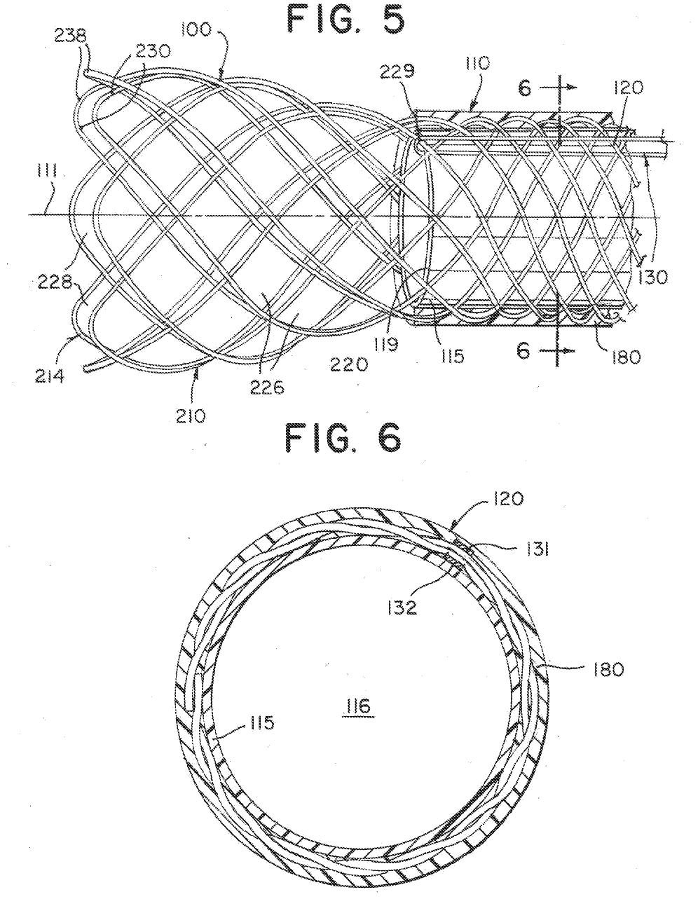

FIG. 5 is similar to FIG. 2, except that the spine hoop 231 illustrated in FIG. 2 is replaced by spine loop 229 that bends approximately 360 degrees near a distal end of the elongate shaft 110 and does not extend into the distal tip section 210. Referring to FIG. 5, spine 130 creates spine loop 229 around the shaft braid and extends only throughout the proximal portion of catheter 100. The strands of spine 130 may be interwoven through the shaft section. Alternatively, one strand may be interwoven and the other strand may be looped back over the braid. The loop may extend around one or more of the braid wires. As illustrated, the loop 229 is positioned at a cross-over point of two braid wires of the shaft braid 120. Alternatively, the spine loop may be free floating and may not loop around any of the braid structure. In some embodiments, tension on the proximal elongate shaft 110 may cause the loop 229 to engage braid wires of the shaft braid 120 at the cross-over point to inhibit elongation of the proximal elongate shaft 110. This example may also potentially be used with fiber strands for the spine 130. The spine may extend a full length of the elongate shaft 110 or only a partial length. The two formed stands 131, 132 may be parallel and one of the parallel strands may be interior of the shaft braid 120. Additionally, one of the parallel stands may be external to the shaft braid 120. The loop 229 can be placed at a variety of locations on the shaft braid 120.

FIG. 6 shows a cross-section of the shaft 110 showing the positioning of the spine 130 of FIG. 5. The inner lumen 116 is contained by inner liner 115. The axial second strand 132 is positioned below the shaft braid 120. The axial first strand 131 is positioned above the shaft braid 120. The outer jacket 180 surrounds the shaft braid 120 and axial second strand 132. The axial first strand 131 and axial second strand 132 may be positioned to be aligned vertically at a local in the shaft braid where the diameter of the braid is minimized (e.g., a cross-over point).

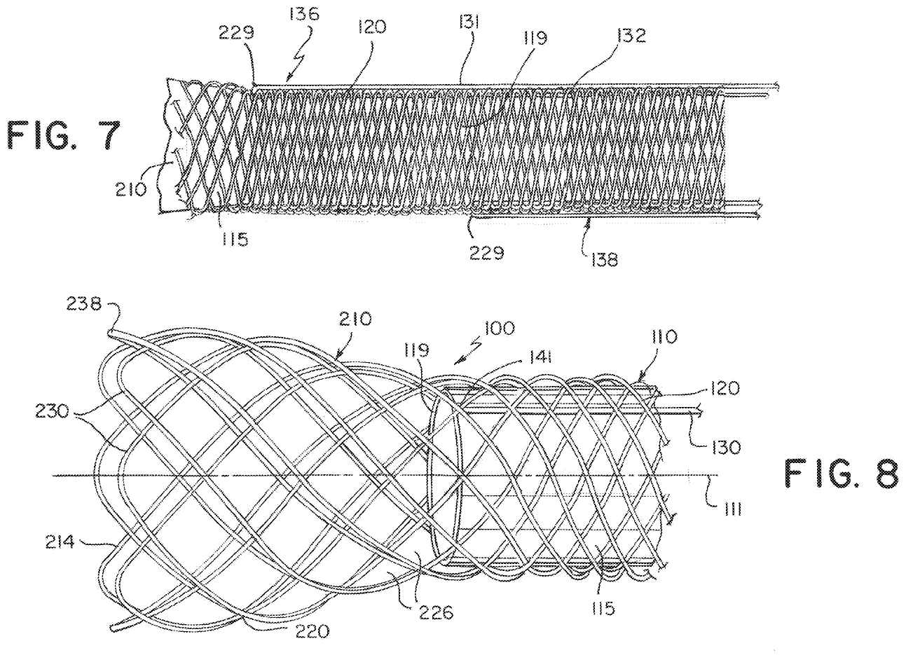

FIG. 7 shows an additional configuration of a catheter with spine reinforcement. FIG. 7 shows a catheter with first spine 136 with axial first strand 131 and axial second strand 132 forming a spine loop 229. The spine loop 229 is positioned where the proximal shaft 110 transitions into the distal tip 210. FIG. 7 also contains a second spine 138 positioned 180 degrees from first spine 136. The second spine 138 only extends just past the distal end 119 of the inner liner 115 such that a spine loop 229 of the second spine 138 is positioned approximate the distal end 119 of the inner liner 115. One, two, three, or more spines may be applied over the braid, under the braid, or interwoven with the braid. The spines may be 180 degrees apart. The addition of two spines will prevent the support tube from lengthening under tensile loading and impart a preferred bending plane on the tube. If aligned parallel to the longitudinal axis, twin spines spaced 180 degrees apart will change the preferred bending place of the tube so that the support tube is capable of self-adjusting as it is advanced through tortuous vessels.

FIG. 8 is similar to FIG. 5, except that the spine 130 has only a single strand. Spine 130 is positioned below the shaft braid 120, and above inner liner 115. Spine 130 only contains a single strand, which ends at termination point 141. Termination point 141 may be located at the cross-over point of the shaft braid. The termination point 141 may be located in a proximal direction in relation to the distal end 119 of the inner liner 115. Alternatively, the single strand may be interwoven. The distal end of the spine may be welded to one or an axial set of braid wires. The distal end may also be tied around one of the braid wires. Alternatively, brazing or an adhesive may be used to hold the distal end of the spine to the braid wires. Either of these methods of fixation prevent the strand from penetrating through the outer jacket.

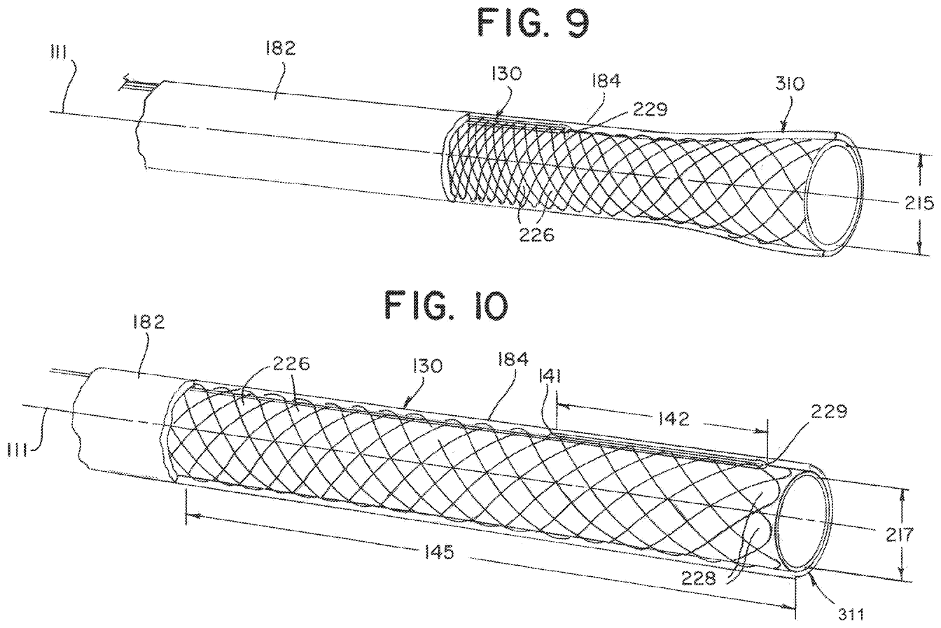

FIG. 9 refers to an additional configuration of a catheter with spine reinforcement. FIG. 9 contains self-expanding collapsible super bore (CSB) tip 310 that has an expanded inner diameter 215. The spine loop 229 extends through a midsection of the of the body. The spine loop 229 may extend halfway along the funnel taper or may extend at the start of the funnel taper. The collapsible super bore catheter may be able to contain an inner catheter with a low shear tip (LST).

FIG. 10 refers to an additional configuration of a catheter with spine reinforcement. FIG. 10 contains a clot-expanding LST tip 311 with an inner diameter 217. Spine 130 extends a majority of the liner distance 145. Spine 130 is a single strand at the proximate end of the catheter, and has a termination point 141 where it forms a loop 229. The spine loop 229 has an inversion length 142 which it extends along the length of the liner. The spine loop 229 goes from below the shaft braid up around the top of the shaft braid to reconnect at the termination point 141. The spine loop 229 may transition from below the shaft braid to above the shaft braid distally of a cross-over of two distal hoops 230. This positioning may be advantageous to prevent lengthening of the LST tip. The spine loop 229 may optionally not reconnect to the lower strands at the termination point 141.

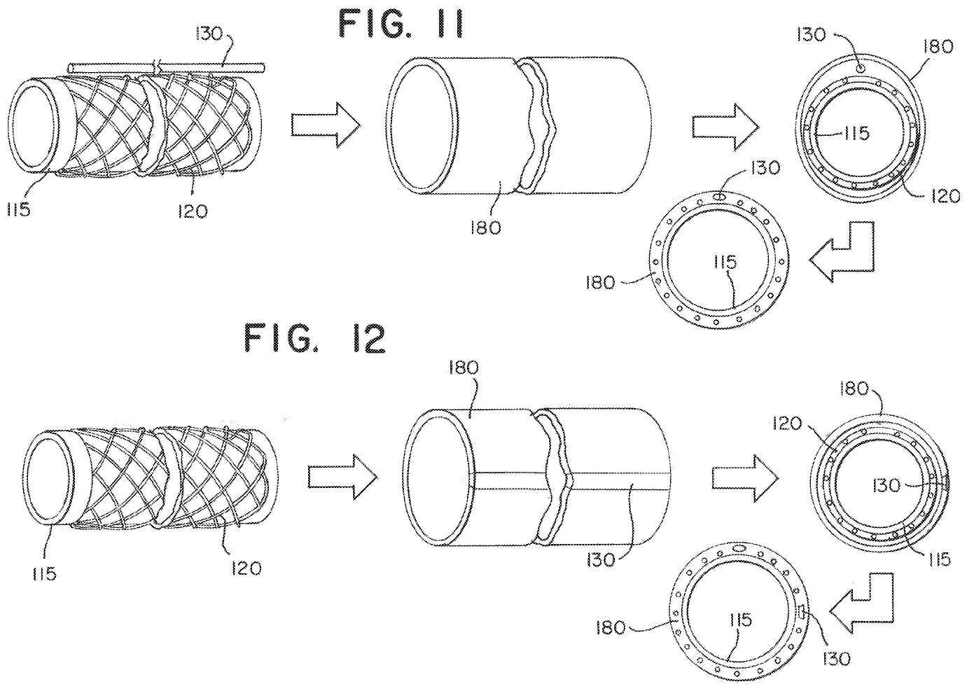

FIG. 11 illustrates a lamination (or reflow) manufacturing method, which can be used to add a polymer spine during the lamination process with minimal impact on flexibility. The polymer spine 130 may have a higher harness than the soft outer jacket 180. The inner liner 115 is located inside of the shaft braid 120. The spine 130 is placed on top of the shaft braid 120 inside of the outer jacket 180. When heated, the outer jacket 180 melts into the shaft braid along with the spine 130. This reflow through the braid allows the braid to bond with the liner, which may be made from PTFE. The spine may be made from Pebax® 40D to 90D or another material, which may be used to increase the force at which tensile elongation occurs for soft outer jackets 180 (may be made from e.g., Neusoft® 42A, 52A, 62A, 72A, Chronoprene® 20A to 80A, ReZalloy 40A to 80A, or ReZithane 40A to 80A). It may be advantageous for the spine to have low elongation relative to the elastomeric outer jacket 180. Polymer spines should have a melt temperature similar to the polymer jackets to improve bonding. The melt temperature of the spine material may be ±40 degrees, ±17 degrees, ±10 degrees, or ±6 degrees Celsius different from the jacket material melt temperature. Ideally, where multiple jackets are used, the melt temperature of the spine most closely matches that of the softer jacket as that section will benefit most from the tensile strength of the spine. For example, nylon may be a good spine material for Neusoft® 42A jackets as they have similar melt temperatures. The nylon also has a higher harness and tensile strength than the Neusoft® 42A, making this a preferable combination.

FIG. 12 illustrates a coextrusion manufacturing method, which can be used to add a polymer spine. The spine 130 is coextruded in the soft outer jacket 180. The shaft braid 120 and inner liner 115 are inserted inside the outer jacket 180 and spine 130. Reflow is then performed over the braid and liner, bonding the outer jacket 180 and spline 130 to the shaft braid 120 and inner liner 115. The spine may be made from Pebax® 60D to 90D or another elastomer, which may be used to increase the force at which tensile elongation occurs for soft outer jackets 180 (may be made from e.g., Neusoft® 42A, 62A, 72A or Chronoprene® 20A to 80A). This has a minimal impact on flexibility.

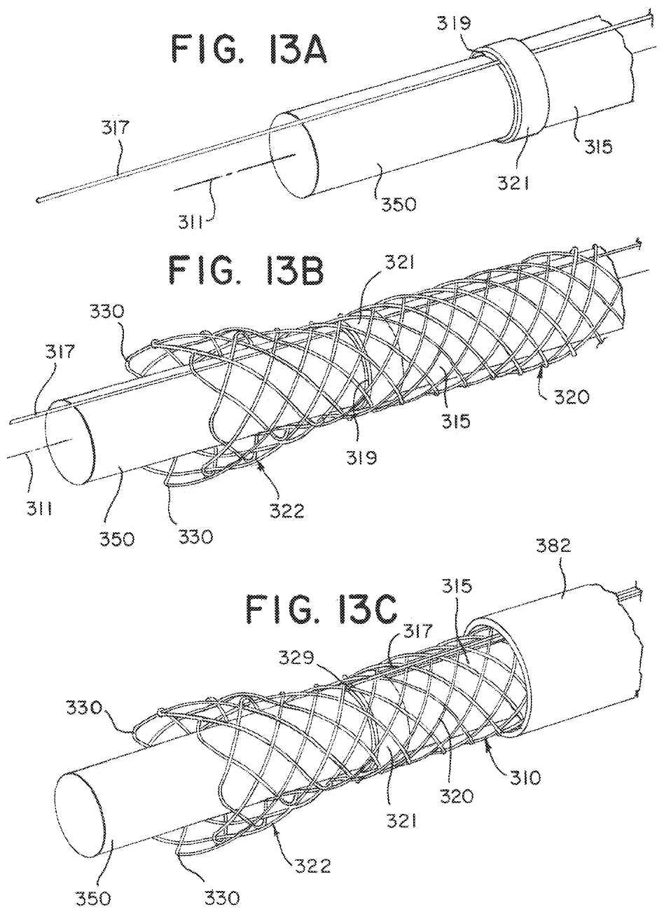

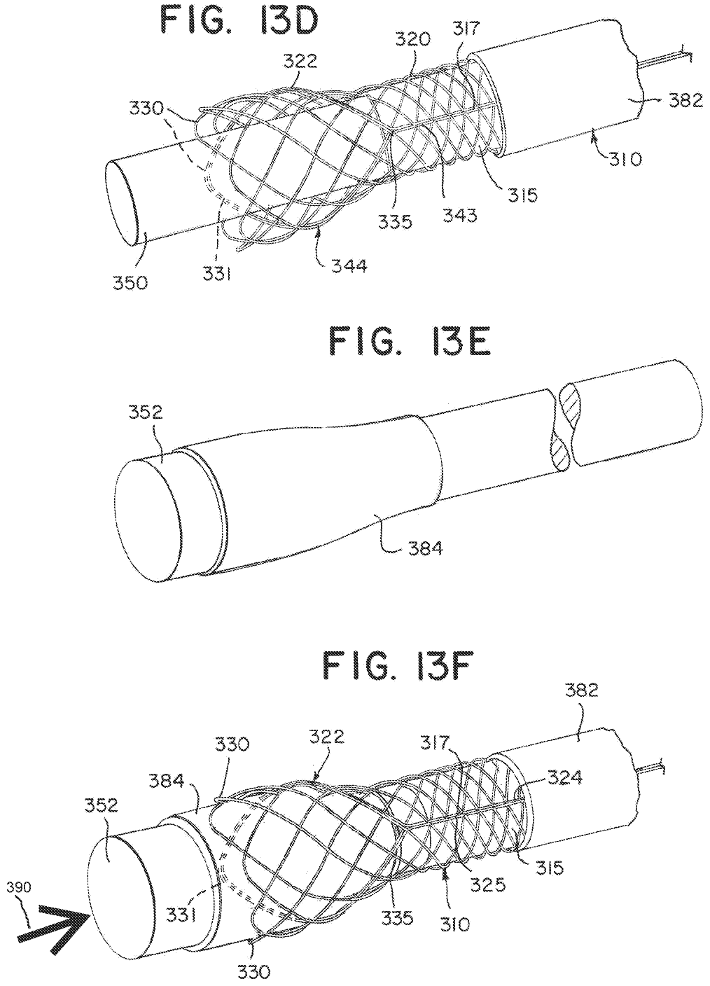

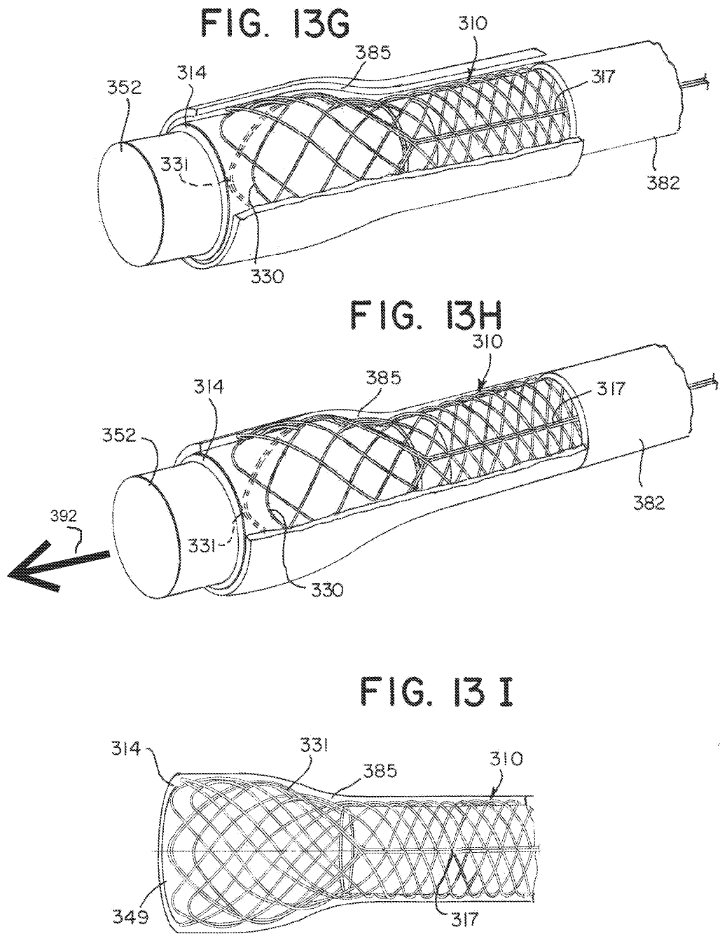

FIGS. 13A-13I show the construction of a catheter with a funnel tip 322. As illustrated in FIG. 13A, construction of this catheter can involve mating the spine 317 and inner liner 315 to braided member 320 using application mandrel 350. Application mandrel 350 contains inner liner 315 over application mandrel 350. Spine 317 is positioned over inner liner 315. At the inner liner distal end 319 the distal marker band 321 is positioned over the top of the inner liner 315 and spine 317.

In FIG. 13B, the application mandrel 350 and spine 317, inner liner 315, and distal marker band 321 are placed inside the braided member 320. FIGS. 13C and 13D show a subsequent construction step for two different spine 130 configurations. FIG. 13C shows the spine 317 folded back to form a spine loop 329 similar to as illustrated in FIG. 5. FIG. 13D shows the spine 317 having a proximal portion 343 and a distal portion 344 separated by a transition point 335 and forming a spine hoop 331 similar to as illustrated in FIG. 2. Alternatively, at the step illustrated in FIGS. 13C and 13D, the spine 317 can be shaped as disclosed elsewhere herein or otherwise shaped as understood by a person skilled in the pertinent art according to the teachings herein. In this step, an outer body jacket 382 is added. In this step, the funnel tip 322 is made by forming braided member 320 into distal hoops 330.

FIG. 13E shows a reflow tool for a funnel tip catheter. This catheter may have a CSB tip. The reflow tool 352 contains the distal inner jacket 384. In FIG. 13F, the reflow tool 352 is inserted into the catheter, which is shown with spine hoop 331, but may also have a spine loop. The distal inner jacket 384 is inserted in the direction of arrow 390 on the inside of the distal hoops 330 for the length of the funnel tip 322 and up to the inner liner 315. In FIG. 13G, the reflow tool 352 is heated, and the distal inner jacket 384 bonds to the distal hoops 330. The distal outer jacket 385 is also placed over the distal end of the catheter. The reflow tool 352 can then be removed in the direction of arrow 392 (shown in FIG. 13H). FIG. 13I is a side view of a completed catheter with mouth 349.

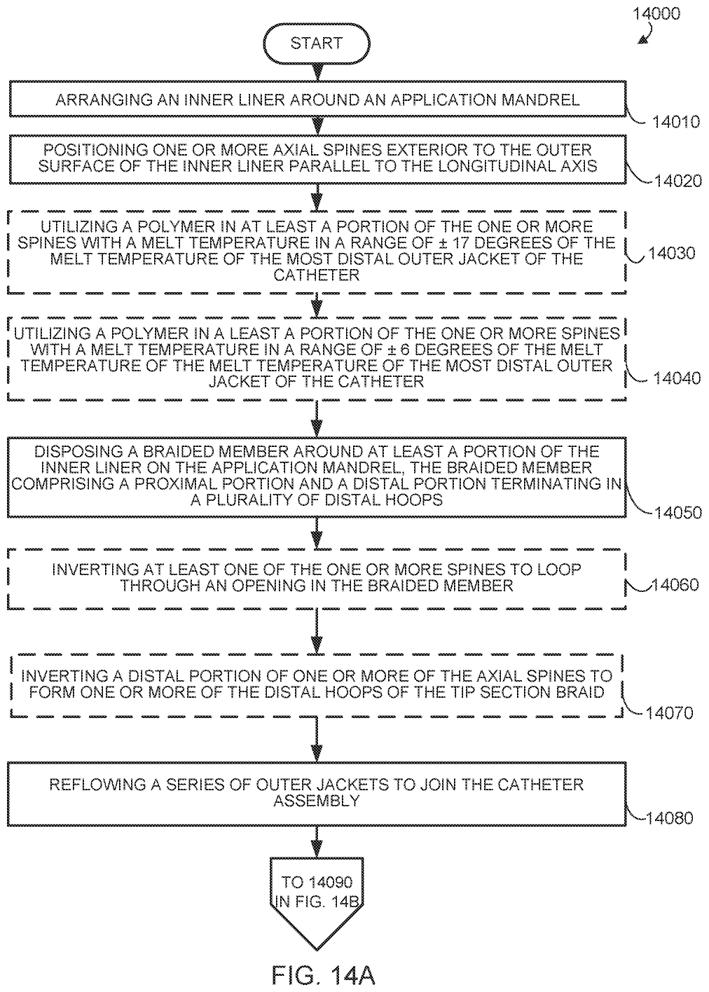

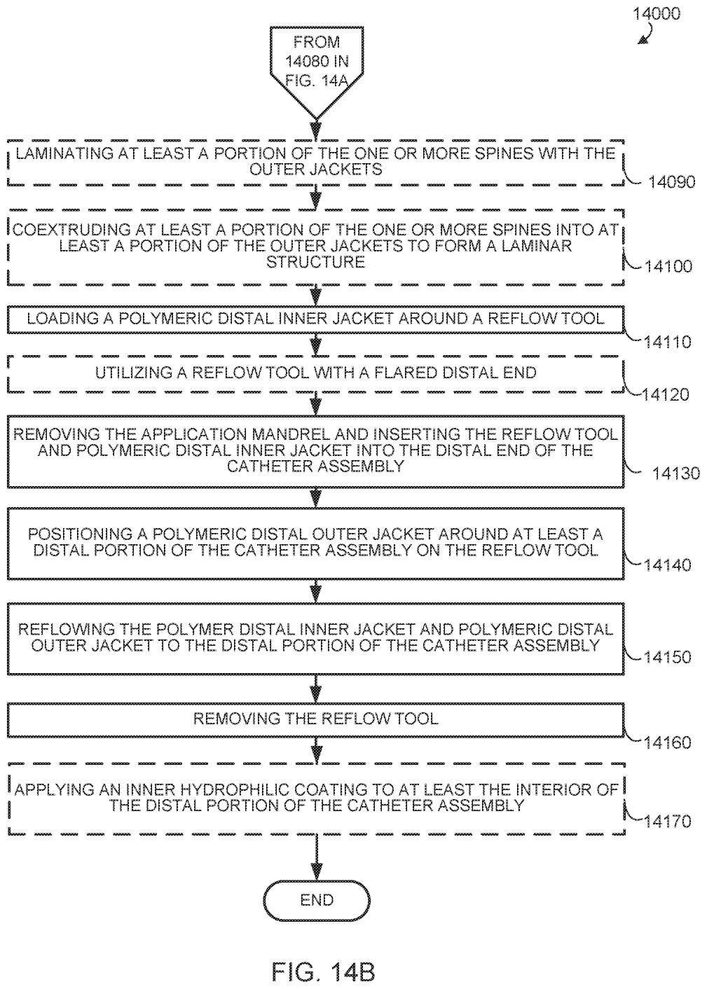

FIGS. 14A and 14B show method 14000 for constructing a catheter with spine reinforcement. In step 14010, the inner liner is arranged around the application mandrel. The mandrel can be used to give structure during manufacturing and define what can be the inner lumen of the catheter. As an examples, a nominal 6Fr size catheter shaft can use an application mandrel with an outer diameter of approximately 0.071 inches, and the inner liner can be approximately 0.005 inches in thickness. The mandrel can be silver-plated copper (SPC) or other commonly used materials and sized to have an outer diameter such that the resulting catheter has a bore larger than many contemporary aspiration catheters (at least 0.070 inches). The liner can be etched PTFE or a similar low friction material. A strike layer can also be included to better adhere the inner liner to subsequent layers of the catheter shaft.

In step 14020, one or more axial spines are positioned exterior to the outer surface of the inner liner and parallel to the longitudinal axis. In optional step 14030, the polymer used for at least a portion of one or more spines has a melt temperature within the range of ±17 degrees Celsius of the melt temperature of the most distal outer jacket of the catheter. Polymer spines may have a melt temperature similar to the polymer jackets to improve bonding. Ideally, where multiple jackets are used, the melt temperature of the spine most closely matches that of the softer jacket as that section will benefit most from the tensile strength of the spine. In optional step 14040, the polymer used for at least a portion of the one or more splines has a melt temperature within the range of ±6 degrees Celsius of the melt temperature of the most distal outer jacket of the catheter. For spines that do not melt or only partially melt, looping the distal end may be advantageous because the spine would be attached to the braid, which would prevent the spine from slipping if the end of the tip was pulled away from the proximal part of the shaft. This additional benefit from looping the distal end may not be present or necessary in spines that completely melt.

In step 14050, a braided member is disposed around at least a portion of the inner liner on the application mandrel. The braided member has a proximal portion and a distal portion. The distal portion terminates in a polarity of distal hoops. This braided member can be the reinforcing structure for most of the catheter shaft. The pattern may utilize 16 wires in a one wire under one over one-half diamond pattern for the tip, as it is balanced during tip expansion. Alternative patterns may include a one wire over two under two herringbone pattern for enhanced column stiffness and kink resistance. Other patterns can also be contemplated for varying requirements of the catheter design (e.g., a full diamond pattern). In optional step 14060, at least one of the one or more spines that loops through an opening in the braided member is inverted. In optional step 14070, the distal portion of one or more of the axial spines are inverted to form one or more of the distal hoops of the tip section braid.

In step 14080, a series of outer jackets are reflowed to join the catheter assembly. The jackets can preferably be in an axial series, but some combination of axial and radial series can be used. The flow of the jacket materials can allow them to encapsulate the braids of the elongate body and bond with the inner liner. In optional step 14090, at least a portion of the one or more spines are laminated with the outer jackets. In optional step 14100, at least a portion of the one or more spines are coextruded into at least a portion of the outer jackets to form a laminar structure.

In step 14110, a polymeric distal inner jacket is loaded around a reflow tool. The supporting braided frame of the distal tip section braid can allow very soft jacket materials to be used here for their atraumatic properties, such as Neusoft with a hardness of 40A to 80A. The inner jacket can be thin (e.g., approximately 0.001 to 0.004 inches) and is used to gain more uniform wall thickness for the tip section and ensure complete encapsulation of the braids, so the lumen is smooth and unobstructed. In optional step 14120, a reflow tool with a flared distal end is utilized. In step 14130, the application mandrel is removed and a reflow tool with a polymeric distal inner jacket is inserted into the distal end of the catheter assembly. The inner jacket may include low friction filler such as Mobilise or Propell to ease removal from the reflow tool and also give low friction to the ID for smooth passing of ancillary devices or more efficient retraction of a clot

In step 14140, the polymeric distal outer jacket is positioned around at least a distal portion of the catheter assembly on the reflow tool. Like the distal inner jacket, the distal outer jacket can be a thin Neusoft layer that can be laminated or reflowed to fuse with the inner jacket such that the final wall thickness of the combined distal polymeric jacket is approximately 0.004 to 0.010 inches, maintaining at least 0.0005 inches of jacket material above and below the braid surfaces. The jacket can extend proximally to the distal edge of the proximal outer jackets of the elongate body and overhang at least several millimetres distally beyond the distal end of the braid hoops of the distal tip braid. In step 14150, the polymer distal inner jacket and polymer distal outer jacket are reflowed to the distal portion of the catheter assembly. In step 14160, the reflow tool is removed.

In optional step 14170, an inner hydrophilic coating is applied to at least the interior of the distal portion of the catheter assembly. A process such as dip coating can be used to apply the coating to both the inner and outer surfaces after removal of the flared mandrel. In most cases the coating can cover at least the distal most 20 cm of the catheter. In some cases, the hydrophilic coating can also be applied to the inner surface of the tip where there is no PTFE liner. Further post-processing, such as reflow and compression/injection moulding steps, can also be used to apply additional material or flow existing material to add features to the expandable tip. The features can be, for example, a feathered edge, a disk extension, a polymeric lip, or axial ribs.

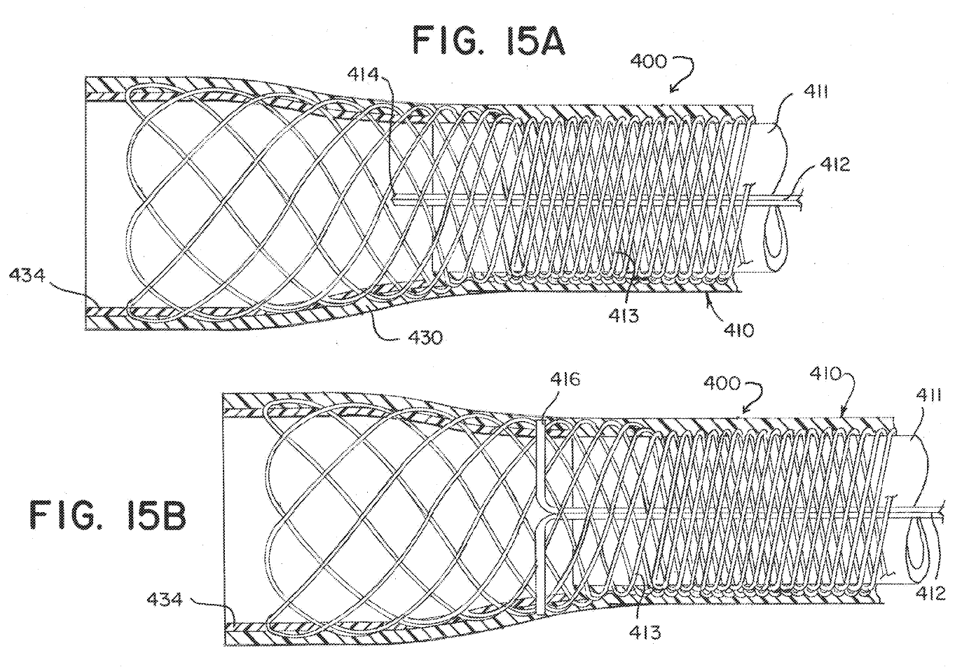

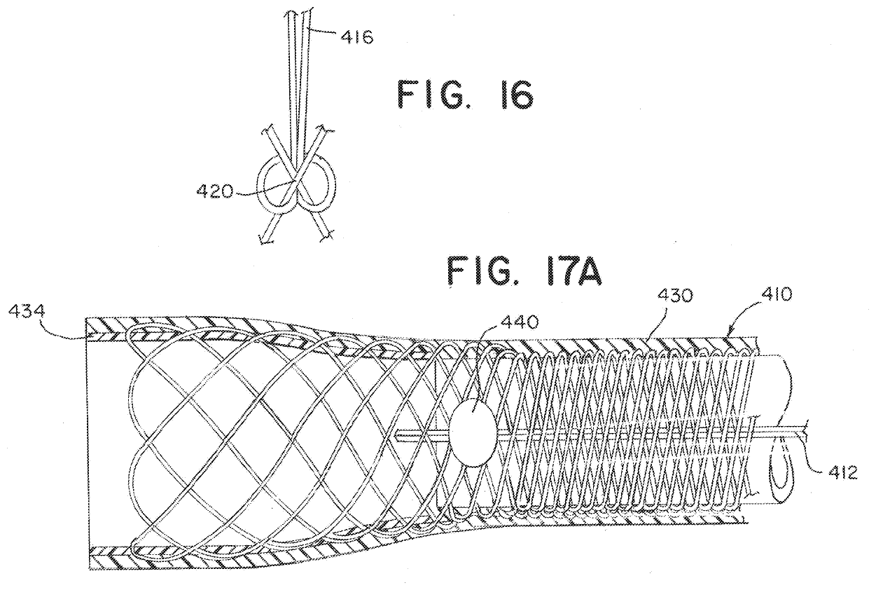

Referring to FIG. 15A, catheter 400 contains elongate body 410, spine 412, distal outer jacket 430, and distal inner jacket 434. Spine 412 has a flat spine end 414 that extends beyond the inner liner 411. The spine 412 is laid under the braid 413 and above the inner liner 411. Alternatively, one end of the spine may be tied to the braid wires to create a distal fixation point. Inner liner 411 may be made of polytetrafluoroethylene (PTFE). The spine material should have sufficient strength to reduce the stretching of the shaft. The spine material may have a strength greater than 5N. The spine material may have a strength greater than 15N. The spine can be metal or a polymer or may be a multifilament polymer.

FIG. 15B is similar to FIG. 15A. However, spine 412 has a lasso spine end 416 that extends outwardly to the distal outer jacket 430. The spine 412 is laid under braid 413 and above the inter liner 411 with a distal loop 416. The distal loop 416 is pulled through a braid opening and around the outer diameter of the braid. Alternatively, the distal loop 416 of the spine 412 can be tied to the braid wires using a lasso knot (shown in FIG. 16). The lasso spine end may be tied to the braid 413 at the braid cross over point 420. Attaching the spine to the braid with a noose or lasso will prevent the braid from stretching and overcoming the adhesion forces between the polymer jackets and the spine.

FIG. 17A is similar to FIG. 15A. In FIG. 17A, a small amount of adhesive 440 is used to fix the distal end of the spine to the braid and liner. FIG. 17B is also similar to FIG. 15A. In FIG. 17B, a short length of higher durometer outer jacket polymer 450 is used to fix the distal end of the spine to the braid and liner. FIG. 17C is also similar to FIG. 15A. In FIG. 17C, a marker band 460 is placed over the braid and spine 412. The marker band 460 may be crimped to secure the spine 412 to the braid, spine, and marker band together. Alternatively, adhesive may be applied under the marker band 460 to fix the braid, spine, and marker band together.

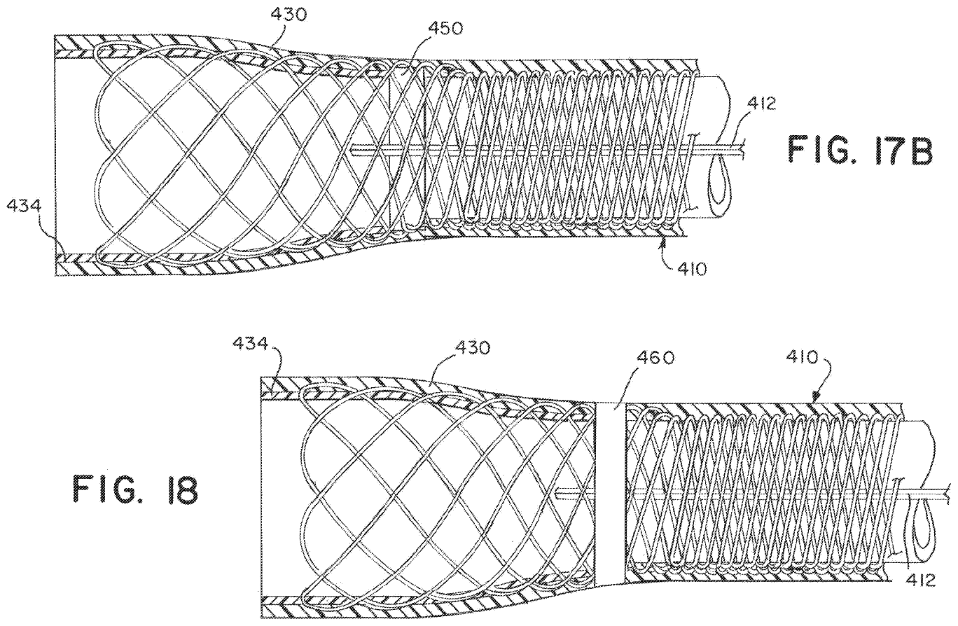

FIG. 18 is a side view of a catheter with spine reinforcement 312 similar to as illustrated in FIGS. 17A and 17B where a marker band 460 is used in attaching the spine and the marker band 460 is over the braid.

Any of the herein disclosed support tubes for clot retrieval catheter designs can be used in conjunction with a mechanical thrombectomy device. The combination of mechanical thrombectomy with aspiration through a funnel-like tip section can increase the likelihood of first pass success in removing a clot. During thrombectomy, a funnel-like shape of the tip section can reduce clot shearing upon entry to the catheter, arrest flow to protect distal vessels from new territory embolization, and also direct the aspiration vacuum to the clot face while the mechanical thrombectomy device will hold a composite clot (comprised of friable regions and fibrin rich regions) together preventing embolization and aid in dislodging the clot from the vessel wall. The shape of the tip can also aid in preventing fragmentation if the clot enters the mouth of the catheter at an offset position.

The mechanical thrombectomy device can be configured to support the lumen of the vessel during aspiration such that it will be less likely to collapse under negative pressure and hold the clot together should the clot comprise an array of stiff and soft portions that may otherwise fragment. The mechanical thrombectomy device can also allow the user to pinch a clot that will not fully enter the lumen of the clot retrieval catheter, thereby ensuring that the clot will not dislodge from the clot retrieval catheter as the clot retrieval catheter, clot, and mechanical thrombectomy device are retracted as one through the vasculature, through the outer catheter, and outside of the patient. The interaction between the outer catheter and the expanded mouth can aid in gradually compressing the clot so that it can be pulled through the outer catheter with the clot retrieval catheter and mechanical thrombectomy device. If the clot is still too large to enter the outer catheter, the clot retrieval catheter and mechanical thrombectomy device can be retracted proximally through the vessel and into a second larger outer catheter such as a balloon guide. Should the clot still be too stiff to retrieve through the second outer catheter, all devices can be retracted together as one through the vasculature and outside of the body. The clot retrieval catheter may be designed to be advanced through an outer catheter such as a 7Fr, 8Fr, 9Fr or 10Fr guide catheter or balloon guide catheter. Alternatively, the clot retrieval catheter may be designed as a 4Fr, 5Fr, 6Fr, intermediate catheter. In another embodiment, the funnel feature may also be provided on a 7Fr, 8Fr, 9Fr or 10Fr guide sheath such that is provides a seal in a larger vessel in place of a balloon guide sheath.

The descriptions contained herein are examples of embodiments of the invention and are not intended in any way to limit the scope of the invention. As described herein, the invention contemplates many variations and modifications of catheters including funnel catheters and tubular aspiration catheters. Modifications and variations apparent to those having skilled in the pertinent art according to the teachings of this disclosure are intended to be within the scope of the claims which follow.

What is claimed is:

1. A catheter comprising:
a proximal elongate shaft comprising a longitudinal axis, a distal end, and a shaft braid;
a distal tip section comprising a tip braid terminating in distal hoops;
a plurality of outer jackets disposed around the elongate shaft and tip section;
one or more metallic spines extending at least partially along an axial length of the catheter, the one or more metallic spines inhibiting tensile elongation of catheter; and
the distal hoops of the tip braid formed monolithically with wires of the shaft braid.

2. The catheter of claim 1, at least a portion of the one or more metallic spines comprising a shape memory alloy.

3. The catheter of claim 1, at least a portion of the one or more metallic spines comprises at least one of stainless steel, DFT, cobalt-chromium, titanium alloy, or tungsten.

4. The catheter of claim 1, at least one of the one or more metallic spines interwoven with the shaft braid.

5. The catheter of claim 1, at least one of the one or more metallic spines comprising a proximal portion and a distal portion, at least a part of the proximal portion comprising two or more adjacent parallel strands.

6. The catheter of claim 5, the distal portion forming one or more of the distal hoops of the tip section braid.

7. The catheter of claim 1, at least a portion of the one or more metallic spines extending the entire axial length of the catheter and comprising a non-circular cross section.

8. The catheter of claim 1, the one or more metallic spines further comprising a first spine and a second spine spaced 180 degrees apart.

9. The catheter of claim 1, at least one of the one or more metallic spines comprising sections of differing thickness and the one or more metallic spines being external to the shaft braid.

10. The catheter of claim 1, the one or more metallic spines further comprising a single spine adhered to one of the shaft braid or the tip braid at a termination point.

11. A catheter comprising:
an elongate tube comprising a longitudinal axis and a shaft braid comprising a first set of helical wires braided with a second set of helical wires;
a distal tip section comprising a tip braid with distal hoops at a distal end;
a plurality of outer jackets disposed around the elongate tube and tip section; and
one or more polymeric spines extending at least partially along an axial length of the catheter, the one or more polymeric spines inhibiting tensile elongation of the catheter;
the first set of helical wires of the shaft braid inverting proximally to form the second set of helical wires of the shaft braid; and
the distal hoops of the tip braid formed monolithically with the wires of the shaft braid.

12. The catheter of claim 11, at least a portion of the one or more polymeric spines comprising a composition of at least one of high-density polyethylene, poly ether ketone, ultra-high molecular weight polyethylene, aromatic polyamide, LCP liquid crystal polymer, Nylon, or thermoset liquid-crystalline polyoxazole.

13. The catheter of claim 11, at least a portion of the one or more polymeric spines being laminated with the outer jackets.

14. The catheter of claim 11, at least a portion of the one or more polymeric spines being coextruded with at least one of the outer jackets.

15. The catheter of claim 11, at least one of the one or more polymeric spines interwoven with the shaft braid.

16. The catheter of claim 11, at least one of the one or more polymeric spines inverting through the shaft braid or the tip braid at a spine loop to form two parallel strands.

17. The catheter of claim 16, at least a portion of one of the parallel strands extending exterior to the shaft braid; and
at least a portion of one of the parallel strands extending interior to the shaft braid.

18. A method for constructing a catheter, the method comprising:
arranging an inner liner around an application mandrel;
positioning one or more axial spines exterior to an outer surface of the inner liner substantially parallel to a longitudinal axis;
disposing a braided member around at least a portion of the inner liner on the application mandrel to form a catheter assembly, the braided member comprising a proximal portion and a distal portion terminating in a plurality of distal hoops;
reflowing a series of proximal outer jackets to join the catheter assembly;
loading a polymeric distal inner jacket around a reflow tool;
removing the application mandrel and inserting the reflow tool and polymeric distal inner jacket into a distal end of the catheter assembly;
positioning a polymeric distal outer jacket around at least the distal portion of the catheter assembly on the reflow tool;
reflowing the polymeric distal inner jacket and polymeric distal outer jacket to the distal portion of the catheter assembly; and
removing the reflow tool.

19. The method of claim 18, further comprising the step of utilizing a polymer in at least a portion of the one or more spines with a melt temperature in a range of approximately ±17 degrees of the melt temperature of the most distal outer jacket of the catheter.

20. The method of claim 19, further comprising the steps of:
inverting at least one of the one or more spines to loop through an opening in the braided member;
inverting a distal portion of one or more of the axial spines to form one or more of the distal hoops of a tip section braid;
laminating at least a portion of the one or more spines with the outer jackets;
coextruding at least a portion of the one or more spines into at least a portion of the outer jackets to form a laminar structure;
utilizing a reflow tool with a flared distal end; and
applying an inner hydrophilic coating at least an interior of the distal portion of the catheter assembly.
